(19) United States
(12) Patent Application Publication
(10) Pub. No.: US 2009/0163801 A1
(43) Pub. Date: Jun. 25, 2009

(54) SYSTEM FOR DISPLAYING DATA RELATING TO ENERGY EMITTING TREATMENT DEVICES TOGETHER WITH ELECTROPHYSIOLOGICAL MAPPING DATA

(75) Inventor: John W. Sliwa, Los Altos Hills, CA (US)

Correspondence Address: SJM/AFD - DYKEMA;c/o CPA Global, P.O. Box 52050, Minneapolis, MN, 55402, US

(73) Assignee: St. Jude Medical, Atrial Fibrillation Division, Inc., Minnetonka, MN (US)

(21) Appl. No.: 11/960,265

(22) Filed: Dec. 19, 2007

(51) Int. Cl. A61B 5/05

(52) U.S. Cl. 600/427

(57) ABSTRACT

A system and method for treatment of tissue is provided. An electronic control unit is configured to generate displays signals used to create a graphical user interface. The display signals are generated in response to a first set of position signals indicative of the position of an electrophysiology mapping electrode relative to the tissue and a second set of position signals indicative of the position of a treatment device, such as a high intensity focused ultrasound transducer, that is configured to generate a beam of energy towards a selected region in the tissue. The graphical user interface displays an electrophysiology map of the tissue and an image of at least one of the treatment device and the beam of energy which may be superimposed on the electrophysiology map.

### BACKGROUND OF THE INVENTION

a. Field of the Invention

The instant invention relates to a system and method for treatment of tissue. In particular, the instant invention relates to a system and method in which at least one of a treatment device and a beam of energy generated by the treatment device during treating of tissue is displayed together with electrophysiological mapping data.

b. Background Art

The use of electrophysiological (EP) mapping data in the diagnosis and treatment of tissues within a body is well known. In a conventional system, a catheter may be inserted within a vessel located near the surface of a body (e.g., in an artery or vein in the leg, neck, or arm) and maneuvered to a region of interest within the body. An electrode disposed at one end of the catheter detects changes in electrical potential resulting from the transmission of electrical signals between points on the body. Signals generated by the electrode are then used to generate an image of a tissue surface.

One existing EP mapping system is sold under the registered trademark “ENSITE” by the assignee of the prevent invention, St. Jude Medical, Atrial Fibrillation Division, Inc. In this system, surface electrode patches are applied in several locations on a body. Electrical signals are transmitted between the patches and one or more electrodes supported within a catheter in the body detect changes in voltage and generate signals that are used to generate an image of a tissue surface. Although the ENSITE system may be used with a variety of conventional catheters and electrodes, the system is preferably used together with the catheter sold by applicant under the registered trademark “ENSITE ARRAY.” This catheter includes multiple electrodes that produce an EP map without requiring contact between the electrodes and the tissue surface.

The EP map generated by the ENSITE system or other conventional mapping systems can be used in the diagnosis and treatment of tissue. For example, EP maps of heart tissue can be used to guide ablation catheters which are used to convey an electrical stimulus to a region of interest within the heart and create tissue necrosis. Ablation catheters may be used to create necrosis in heart tissue to correct conditions such as atrial arrhythmia (including, but not limited to, ectopic atrial tachycardia, atrial fibrillation, and atrial flutter). Arrhythmia can create a variety of dangerous conditions including irregular heart rates, loss of synchronous atrioventricular contractions and statis of blood flow which can lead to a variety of ailments and even death. It is believed that the primary cause of arrhythmia is stray electrical signals within the left or right atrium of the heart. The ablation catheter imparts ablative energy (e.g., radiofrequency energy) to the heart tissue to create a lesion in the heart tissue. This lesion disrupts electrical pathways and thereby limits or prevents stray electrical signals that lead to arrhythmia.

The ENSITE system and other mapping systems generate a display containing the EP map and may also display the catheter and/or mapping electrode used in generating the map. The ENSITE system conveys information regarding the location and orientation of the catheter and whether the catheter is in contact with the tissue.

The inventor herein has recognized a need for a system and method for treatment of tissue that will not only provide information regarding the location and orientation of the mapping electrode, but that will also provide information regarding treatment devices used during treatment of tissue.

### BRIEF SUMMARY OF THE INVENTION

It is desirable to be able to provide information to physicians involved in the diagnosis and treatment of tissues including the position and state of the tissue and information regarding diagnostic and/or treatment devices used during diagnosis and treatment of the tissue. For example, during treatment of atrial arrhythmia, it would be useful to know not only the position of an electrophysiological mapping electrode, but also the position and orientation of a treatment device such as an ablation catheter and the ablative energy emanating from the device. This information would assist the physician in creating a lesion that is effective in treating the arrhythmia while minimizing potential risks to the patient. The inventor has therefore developed a system and method for treatment of tissue.

A system for treatment of tissue in accordance with one aspect of the present invention includes an electronic control unit configured to generate display signals used to generate a graphical user interface. The display signals are generated in response to a first set of position signals generated by an electrophysiology mapping electrode that are indicative of a position of the electrode relative to the tissue. The display signals are also generated in response to a second set of position signals indicative of a position of a treatment device. The treatment device is configured to generate and direct a beam of energy towards a selected region in the tissue. This energy may, for example, comprise electromagnetic radiation and the treatment device may comprise, for example, a high intensity focused ultrasound transducer. The graphical user interface displays an electrophysiology map of the tissue and an image of at least one of the treatment device and the beam of energy.

A method in accordance with one aspect of the present invention may include the step of positioning an electrophysiology mapping electrode relative to a tissue in a body. The electrode generates a first set of position signals indicative of a position of the electrode relative to the tissue. The method may further include the step of positioning a treatment device relative to the tissue, the treatment device configured to direct a beam of energy towards a selected region in the tissue. The method may further include the step of generating display signals in response to the first set of position signals and a second set of position signals indicative of a position of the treatment device. The method may further include the step of generating a graphical user interface responsive to the display signals, the graphical user interface displaying an electrophysiology map of the tissue and an image of at least one of the treatment device and the beam of energy.

The above-described system and method are advantageous because they provide significant information to the physician regarding the location and orientation of the device being used to treat the tissue as well as the energy emanating from the device. Using this information, the physician can provide more effective treatment with less risk to the patient.

The foregoing and other aspects, features, details, utilities and advantages of the present invention will be apparent from reading the following description and claims, and from reviewing the accompanying drawings.

### BRIEF DESCRIPTION OF THE DRAWINGS

FIG. 1 is diagrammatic view of a system in accordance with one embodiment of the present invention.

FIG. 2 is a diagrammatic view illustrating an electrophysiological catheter and an energy emitting treatment device during treatment of cardiac tissue.

FIG. 3 is a diagrammatic view of a display screen illustrating a graphical user interface generated in accordance with the present invention.

### DETAILED DESCRIPTION OF EMBODIMENTS OF THE INVENTION

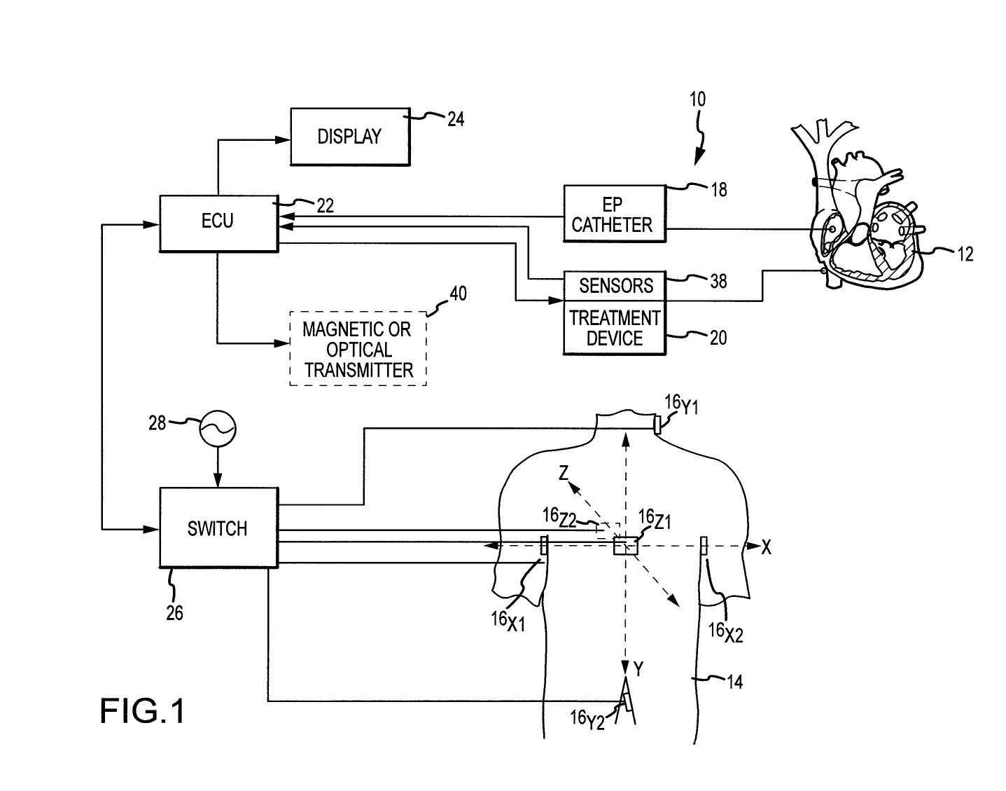

Referring now to the drawings wherein like reference numerals are used to identify identical components in the various views, FIG. 1 illustrates a system 10 for treatment of tissue 12 in a body 14. In one embodiment of the invention, tissue 12 comprises heart tissue within a human body. It should be understood, however, that the inventive system 10 may find application in connection with the treatment of a variety of tissues within human and non-human bodies. System 10 may include a plurality of patch electrodes 16 applied to body 14, an electrophysiological (EP) catheter 18, a treatment device 20, an electronic control unit (ECU) 22 and a display 24.

Patch electrodes 16 are provided to generate electrical signals used in determining the position of catheter 18 and in generating EP data regarding tissue 12. Electrodes 16 may also be used in determining the position of treatment device 20 and related information. Electrodes 16 are placed orthogonally on the surface of body 14 and are used to create axes specific electric fields within body 14. Electrodes 16_{X1}, 16_{X2} may be placed along a first (x) axis. Similarly, electrodes 16_{Y1}, 16_{Y2} may be placed along a second (y) axis and electrodes 16_{Z1}, 16_{Z2} may be placed along a third (z) axis. Each of the electrodes 16 may be coupled to a multiplex switch 26. ECU 22 is configured through appropriate software to provide control signals to switch 26 and thereby sequentially couple pairs of electrodes 16 to a signal generator 28. Excitation of each pair of electrodes 16 generates a magnetic field within body 14 and within an area of interest such as tissue 12. Voltage levels at non-excited electrodes 16 are filtered and converted and provided to ECU 22 for use as reference values.

EP catheter 18 is provided for use in gathering EP data associated with tissue 12 to enable generation of an image of the geometry of the tissue surface and related EP data. Catheter 18 may also allow removal of bodily fluids or injection of fluids and medicine into the body and may further provide a means for transporting surgical tools or instruments within a body. Catheter 18 may be formed from conventional materials such as polyurethane. Catheter 18 is tubular and is deformable and may be guided within a body by a guide wire or other means known in the art. Catheter 18 has a proximal end and a distal end (as used herein, “proximal” refers to a direction toward the body of a patient and away from the physician while “distal” refers to a direction toward the physician and away from the body of a patient). Catheter 18 may be inserted within a vessel located near the surface of a body (e.g., in an artery or vein in the leg, neck, or arm) in a conventional manner and maneuvered to a region of interest in body 14 such as tissue 12.

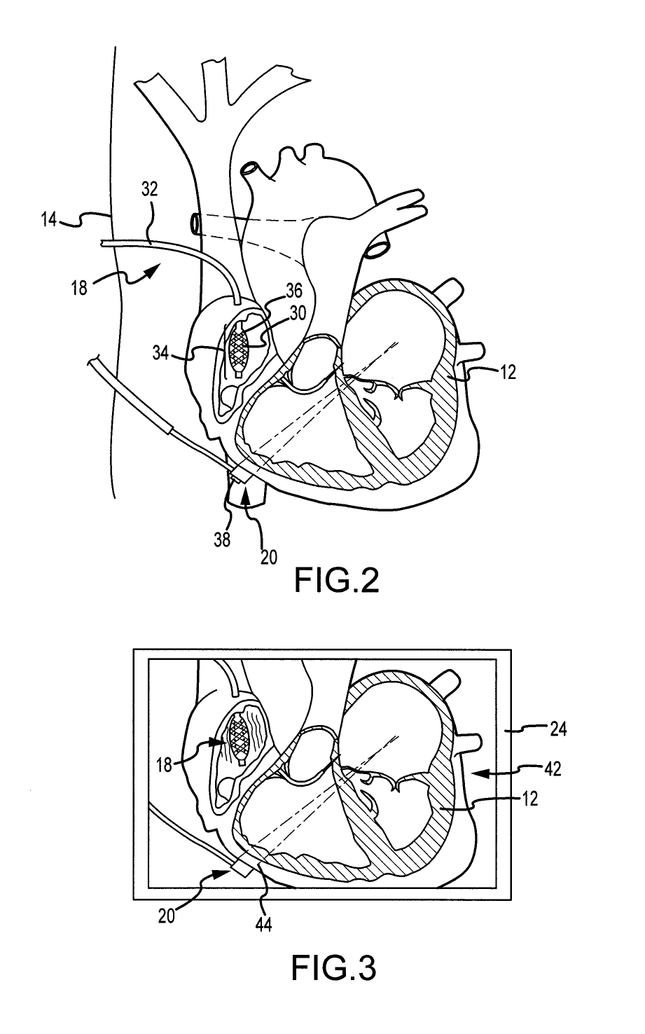

Referring to FIG. 2, EP catheter 18 includes a plurality of EP mapping electrodes 30. The electrodes 30 are placed within electrical fields created in body 14 (e.g., within the heart) by exciting patch electrodes 16. The electrodes 30 experience voltages that are dependent on the location between the patch electrodes 16 and the position of the electrodes 30 relative to tissue 12. Voltage measurement comparisons made between electrodes 30 can be used to determine the position of the electrodes 30 relative to tissue 12. Movement of the electrodes 30 proximate tissue 12 (e.g., within a heart chamber) produces information regarding the geometry of the tissue 12 as well as EP data. For example, voltage levels on the tissue surface over time may be projected on the image of the geometry of the tissue as an activation map. The voltage levels may be represented in various colors and the EP data may be animated to show the passage of electromagnetic waves over the tissue surface. Information received from the electrodes 30 can also be used to display the location and orientation of the electrodes 30 and/or the tip of EP catheter 18 relative to tissue 12.

EP catheter 18 is preferably a non-contact mapping catheter such as the catheter sold by St. Jude Medical, Atrial Fibrillation Division, Inc. under the registered trademark “ENSITE ARRAY.” It should be understood, however, that the present invention may also be used with contact mapping systems in which measurements are taken through contact of the electrodes with the tissue surface. Referring to FIG. 2, catheter 18 includes a deformable tubular body 32 including a deformable distal portion 34. Portion 34 may be formed as a braid of insulated wires 36 with an array of electrodes 30 formed where the insulation on the wires 36 has been removed. Portion 34 may be deformed by expansion (e.g. through use of a balloon) into a stable and reproducible geometric shape to fill a space (e.g., a portion of a heart chamber) after introduction into the space. One or more reference electrodes (not shown) may also be located nearer the distal end of catheter 18 than electrodes 30 and may contact the tissue surface to calibrate the electrode array and maintain the position of the electrode array. An exemplary EP catheter is shown in commonly assigned U.S. Pat. No. 7,289,843, the entire disclosure of which is incorporated herein by reference.

Treatment device 20 is used to perform surgical procedures on tissue 12 and/or to deliver drugs or medicine to tissue 12. Treatment device 20 may, for example, comprise an ablation catheter. It should be understood, however, that the present invention could be used with a wide variety of surgical and drug delivery devices. The present invention has particular use in connection with treatment devices that treat tissue 12 by emitting a beam of energy such as ablation catheters. The emitted energy may comprise electromagnetic radiation including, for example, a laser. In one embodiment of the invention device 20 comprises a high intensity focused ultrasound (HIFU) transducer.

Referring to FIGS. 1-2, treatment device 20 includes means, such as one or more position sensors 38, for determining the position and orientation of treatment device 20. A variety of conventional position sensors 38 could be used. Sensors 38 may comprise one or more electrodes that work in a manner similar to electrodes 30 in EP catheter 18—generating signals responsive to excitation of patch electrodes 16 that permit ECU 22 to determine the location of treatment device 20. In another embodiment, system 10 may include means, such as a transmitter 40, for providing a stable reference signal detected by sensors 38. In one embodiment, the position sensor 38 may include three wire coils arranged orthogonally to one another. Referring to FIG. 1, transmitter 40 may also include three wire coils arranged orthogonally to one another. ECU 22 may sequentially pulse the coils in transmitter 40 and obtain corresponding vector components from the coils in sensors 38 from which a distance and orientation relative to the reference transmitter 40 can be determined. In another embodiment, the position sensors 38 may—in certain applications—comprise optical sensors (e.g., a photodiode) while transmitter 40 comprises an optical transmitter (e.g., a light emitting diode). Signals generated by the optical sensors in response to the intensity of the received electromagnetic radiation, may enable ECU 22 to determine the position and orientation of treatment device 20.

Electronic control unit (ECU) 22 provides a means for generating display signals used to control display 24 and the creation of a graphical user interface (GUI) on display 24. ECU 22 also provides a means for determining the geometry of tissue 12, EP characteristics of tissue 12 and the position and orientation of EP catheter 18 and treatment device 20 as well as the path of energy emitted from treatment device 20. ECU 22 may further provide a means for controlling various components of system 10 including, but not limited to, treatment device 20, switch 26 and transmitter 40. ECU 22 may comprise a programmable microprocessor or microcontroller or may comprise an application specific integrated circuit (ASIC). ECU 22 may include a central processing unit (CPU) and an input/output (I/O) interface through which ECU 22 may receive a plurality of input signals including signals generated by patch electrodes 16, EP catheter 18 (and mapping electrodes 30), and position sensors 38 and generate a plurality of output signals including those used to control and/or provide data to treatment device 20, display 24, switch 26 and transmitter 40.

In operation, ECU 22 generates signals to control switch 26 and thereby selectively energize patch electrodes 16. ECU 22 receives position signals from EP catheter 18 (and particularly mapping electrodes 30) reflecting changes in voltage levels on mapping electrodes 30 and from the non-energized patch electrodes 16. ECU 22 uses the raw location data produced by electrodes 16, 30 and corrects the data to account for respiration and other artifacts. ECU 22 then generates display signals to create an electrophysiological map of tissue 12. ECU 22 also receives position signals from position sensors 38 on treatment device 20. ECU 22 uses the raw location data produced by sensors 38 and again corrects the data to account for respiration and other artifacts. ECU 22 then generates display signals to create an image of treatment device 20 and/or the energy emitted by device 20 that may be superimposed on the EP map. Because the path and other characteristics of the beam of energy generated by device 20 are known based on controlled inputs to device 20, knowledge of the location and orientation of device 20 permits a reliable reproduction of the beam of energy on display 24. Moreover, the image of device 20 and/or the beam of energy on display 24 indicates to the treating physician the location in tissue 12 in which the energy will be deposited.

Display 24 is provided to convey information to a physician to assist in diagnosis and treatment of tissue 12. Display 24 may comprise a conventional computer monitor or other display device. Display 24 presents a graphical user interface (GUI) 42 to the physician. The GUI 42 may include a variety of information (most of which is not shown in FIG. 3) including, for example, an image of the geometry of the tissue, EP data associated with tissue 12, graphs illustrating voltage levels over time for various electrodes, and images of EP catheter 18 and mapping electrodes 30. Examples of the type of information that may be displayed are shown in commonly assigned U.S. Pat. No. 7,263,397, the entire disclosure of which is incorporated herein by reference.

Referring to FIG. 3, in accordance with the present invention, the GUI 42 on display 24 displays both an electrophysiology map of tissue 12 and an image of at least one of treatment device 20 and the beam 44 of energy emitted by device 20. The EP map preferably includes both the geometry of the tissue and EP data associated with the tissue. For example, the EP map may include an image of tissue 12 together with color coded indicators illustrating electrical activity at locations in tissue 12. The EP map may comprise a two-dimensional image of tissue 12 (e.g., a cross-section of the heart) or a three-dimensional image of tissue 12. Display 24 may also include an image of the EP catheter 18 and/or mapping electrodes 30 illustrating their position relative to tissue 12. As discussed hereinabove, the use of position sensors 38 on treatment device allows the position and orientation of device 20 relative to tissue 12 and within body 14 to be determined. As a result, the GUI 42 may include an image of device 20 which may be superimposed onto the EP map. Furthermore, because the path and other characteristics of the beam 44 of energy emitted from device 20 are known (based on known inputs used to generate beam 44) and the location and orientation of device 20 are known, GUI 42 may also include a reliable image of beam 44. This image may also be superimposed onto the EP map. Based on the image of device 20 and/or the beam 44 of energy, the treating physician can identify the location in tissue 12 in which the energy will be deposited.

In practice, a method of treating tissue in accordance with the present invention may include several steps. One or more electrophysiology (EP) mapping electrodes 30 may be positioned relative to the tissue 12 in body 14. The electrodes 30 may be positioned by inserting a catheter 18 into a vessel near the surface of the body and navigating the catheter 18 (e.g., through the use of a fluoroscope) to a region of interest such as tissue 12. The mapping electrode(s) 30 will generate a set of position signals indicative of a position of the electrode relative to tissue 12. The method may also the step of positioning a treatment device 20 relative to the tissue 12. Treatment device 20 may be positioned by inserting device 20 through the same or a different vessel and navigating device 20 to a region of interest such as tissue 12. The method may further include the step of generating display signals in response to the position signals generated by electrode(s) 30 and position signals indicative of the position of treatment device 20 relative to tissue 12. As discussed above, the position of treatment device 20 may be determined using position sensors 38 affixed to device 20. ECU 22 may use the signals from electrodes 30 and sensors 38 (as well as other information from other sources including the non-excited patch electrodes 16) to generate the display signals used to power display. The method may therefore conclude with the step of generating a graphical user interface 42 responsive to the display signals with the graphical user interface 42 displaying both an EP map of tissue 12 and an image of at least one of device 20 and the beam 44 of energy emitted by device 20.

A system and method in accordance with the present invention offer a number of advantages. Because the inventive system and method provide information relating to the treatment device (and energy emitted by the treatment device) with the EP map, greater accuracy and efficiency in treatment can be obtained. A treating physician is able to better pinpoint the location for treatment as well as assess treatment in real time thereby reducing risk to patients.

Although several embodiments of this invention have been described above with a certain degree of particularity, those skilled in the art could make numerous alterations to the disclose embodiments without departing from the spirit or scope of this invention. All directional references (e.g., upper, lower, upward, downward, left, right, leftward, rightward, top, bottom, above, below, vertical, horizontal, clockwise and counterclockwise) are only use for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not as limiting. Changes in detail or structure may be made without departing from the spirit of the invention as defined in the appended claims.

What is claimed is:

1. A system for treatment of a tissue in a body, comprising:
an electronic control unit configured to generate display signals used to generate a graphical user interface in response to a first set of position signals generated by an electrophysiology mapping electrode, said position signals indicative of a position of said electrode relative to said tissue, and in response to a second set of position signals indicative of a position of a treatment device, said treatment device configured to generate and direct a beam of energy towards a selected region in said tissue,
wherein said graphical user interface displays an electrophysiology map of said tissue and an image of at least one of said treatment device and said beam of energy.

2. The system of claim 1 wherein said tissue comprises cardiac tissue.

3. The system of claim 1 wherein said electrophysiology map comprises a two-dimensional image of said tissue.

4. The system of claim 1 wherein said electrophysiology map comprises a three-dimensional image of said tissue.

5. The system of claim 1 wherein said energy comprises electromagnetic radiation.

6. The system of claim 5 wherein said electromagnetic radiation comprises ultrasound.

7. The system of claim 1 wherein said image of at least one of said treatment device and said beam of energy is superimposed on said electrophysiology map.

8. The system of claim 1 wherein said mapping electrode is a non-contact mapping electrode.

9. The system of claim 1 wherein said treatment device comprises a high intensity focused ultrasound transducer.

10. The system of claim 1 wherein second set of position signals are generated by a position sensor coupled to said treatment device.

11. A method for treatment of tissue in a body, comprising the steps of:
positioning an electrophysiology mapping electrode relative to a tissue in a body, said electrode generating a first set of position signals indicative of a position of said electrode relative to said tissue;
positioning a treatment device relative to said tissue, said treatment device configured to direct a beam of energy towards a selected region in said tissue;
generating display signals in response to said first set of position signals and a second set of position signals indicative of a position of said treatment device;
generating a graphical user interface responsive to said display signals, said graphical user interface displaying an electrophysiology map of said tissue and an image of at least one of said treatment device and said beam of energy.

12. The method of claim 11 wherein said tissue comprises cardiac tissue.

13. The method of claim 11 wherein said electrophysiology map comprises a two-dimensional image of said tissue.

14. The method of claim 11 wherein said electrophysiology map comprises a three-dimensional image of said tissue.

15. The method of claim 11 wherein said energy comprises electromagnetic radiation.

16. The method of claim 11 wherein said electromagnetic radiation comprises ultrasound.

17. The method of claim 11 wherein said image of at least one of said treatment device and said beam of energy is superimposed on said electrophysiology map.

18. The method of claim 11 wherein said mapping electrode is a non-contact mapping electrode.

19. The method of claim 11 wherein said treatment device comprises a high intensity focused ultrasound transducer.

20. The method of claim 11 wherein second set of position signals are generated by a position sensor coupled to said treatment device.
